(19) United States
(12) Patent Application Publication
(10) Pub. No.: US 2016/0000956 A1
(43) Pub. Date: Jan. 7, 2016

(54) CONTROLLED INTERMITTENT FRAGRANCE DELIVERY DEVICES AND CARTOMIZER CARTRIDGE FOR USE THEREIN

(71) Applicant: W. McJames, LLC, Bedford, NH (US)

(72) Inventors: Donald JENKINS, Nashua, NH (US); David ALTOBELLI, Hollis, NH (US); Thomas DAVIS, Hollis, NH (US)

(21) Appl. No.: 14/789,274

(22) Filed: Jul. 1, 2015

Related U.S. Application Data
(60) Provisional application No. 62/019,567, filed on Jul. 1, 2014.

(51) Int. Cl. A61L 9/03; H05B 1/02

(52) U.S. Cl. CPC A61L 9/037; A61L 9/032; H05B 1/02; H05B 1/0297; H05B 2203/021; H05B 2203/035

(57) ABSTRACT

Fragrance delivery devices, consistent with embodiments of the present disclosure, may be controlled to deliver a fragrance intermittently and/or selectively. A fragrance delivery device generally includes a housing containing a cartomizer for producing a fragrance vapor from a fragrance liquid, and control circuitry for controlling the production of the fragrance vapor in response to a user input. The fragrance delivery device may include a user interface and/or a wireless interface for connecting to a remote control device, such as a smart phone, tablet, or other computing device, to provide the user input. The fragrance delivery device may deliver the fragrance vapor using natural convection and/or by moving air through the device, for example, with a fan. The fragrance delivery device may also include a power source, such as a battery, and/or may include a connection to an external power source.

This application claims benefit of U.S. Provisional Patent Application No. 62/019,567, titled “Controlled Intermittent Fragrance Delivery Devices and Cartomizer Cartridges For use Therein,” filed on Jul. 1, 2014, the entire disclosure of which is incorporated herein by reference.

### TECHNICAL FIELD

The present disclosure relates generally to fragrance delivery devices, and more particularly, to a controlled intermittent fragrance delivery device.

### BACKGROUND INFORMATION

Olfactory technology continues to grow in popularity and application. For example, scent-generating devices that produce one or more fragrances are well suited for aroma therapy, controlling or otherwise masking unpleasant odors, and enhancing one's personal scent. Such devices may derive power from a battery source and, as a result, offer portable scent-generating solutions. However, the effects of olfactory fatigue or adoption, which is an example of neural or sensory adaption, can reduce the efficacy of such devices. This is because prolonged exposure to an airborne compound causes a neural response that reduces the brain's ability to distinguish a particular odor or scent. For example, a person who wears perfume or cologne stops perceiving or otherwise enjoying a selected scent moments after application, while others who intermittently interact with the person continue to perceive the scent.

### BRIEF DESCRIPTION OF THE DRAWINGS

These and other features and advantages will be better understood by reading the following detailed description, taken together with the drawings wherein:

FIG. 1 shows one example of a fragrance delivery device, consistent with an embodiment of the present disclosure.

FIG. 2 shows another example of a fragrance delivery device, consistent with an embodiment of the present disclosure.

FIG. 3 shows a top perspective view of a fragrance delivery device, consistent with an embodiment of the present disclosure.

FIG. 4 illustrates an end view of the fragrance delivery device shown in FIG. 3.

FIG. 5 illustrates a side cross-sectional view of the fragrance delivery device taken along line A-A in FIG. 4.

FIG. 6 shows a top view of the fragrance delivery device of FIG. 3.

FIG. 7 shows a perspective view of a covered fragrance delivery device, consistent with another embodiment of the present disclosure.

FIG. 8 shows a side view of the covered fragrance delivery device of FIG. 7.

FIG. 9 shows an exploded view of one example removable cartomizer cartridge for use in a fragrance delivery device, in accordance with an embodiment of the present disclosure.

FIG. 10 shows one example method for receiving and executing a fragrance delivery program to selectively control the production of fragrance vapor by a fragrance delivery device.

FIG. 11 shows one example timing diagram and illustrates a plurality of signals configured to selectively energize one or more cartomizer cartridges during execution of the method of FIG. 10, in accordance with an embodiment of the present disclosure.

### DETAILED DESCRIPTION

Fragrance delivery devices, consistent with embodiments of the present disclosure, may be controlled to deliver a fragrance intermittently and/or selectively. A fragrance delivery device generally includes a housing containing a cartomizer for producing a fragrance vapor from a fragrance liquid, and control circuitry for controlling the production of the fragrance vapor in response to a user input. The fragrance delivery device may include a user interface and/or a wireless interface for connecting to a remote control device, such as a smart phone, tablet, or other computing device, to provide the user input. The fragrance delivery device may deliver the fragrance vapor using natural convection and/or by moving air through the device, for example, with a fan. The fragrance delivery device may also include a power source, such as a battery, and/or may include a connection to an external power source.

The cartomizer generally includes a reservoir for containing a fragrance liquid, a wick for receiving the fragrance liquid, and a heating coil around at least a portion of the wick for heating the fragrance liquid to produce the fragrance vapor. The control circuitry receives a user input and generates a control signal, in response to the user input, for controllably energizing the heating coil in the cartomizer to intermittently heat the fragrance liquid and intermittently produce the fragrance vapor. The control circuitry may include any combination of hardware, software, and firmware capable of performing the functions described herein.

The cartomizer may produce the fragrance vapor by converting volatile substances into a vapor or aerosol, for example, using techniques known for use in e-cigarettes. The control circuitry may be used to control the production of the fragrance vapor, for example, in a manner that minimizes the effects of olfactory fatigue or adaption. The control circuitry may be used to adjust the intensity of the fragrance, for example, by using pulse width modulation of the control signal and/or by variable convection (e.g., by controlling a fan). The control circuitry may also be used to introduce intervals or gaps in the presentation of the scent stimulus to allow the olfactory sensory system to “reset” and then be able to continue to perceive and enjoy the scent. Thus, individuals may be able to enjoy selected scents more continuously.

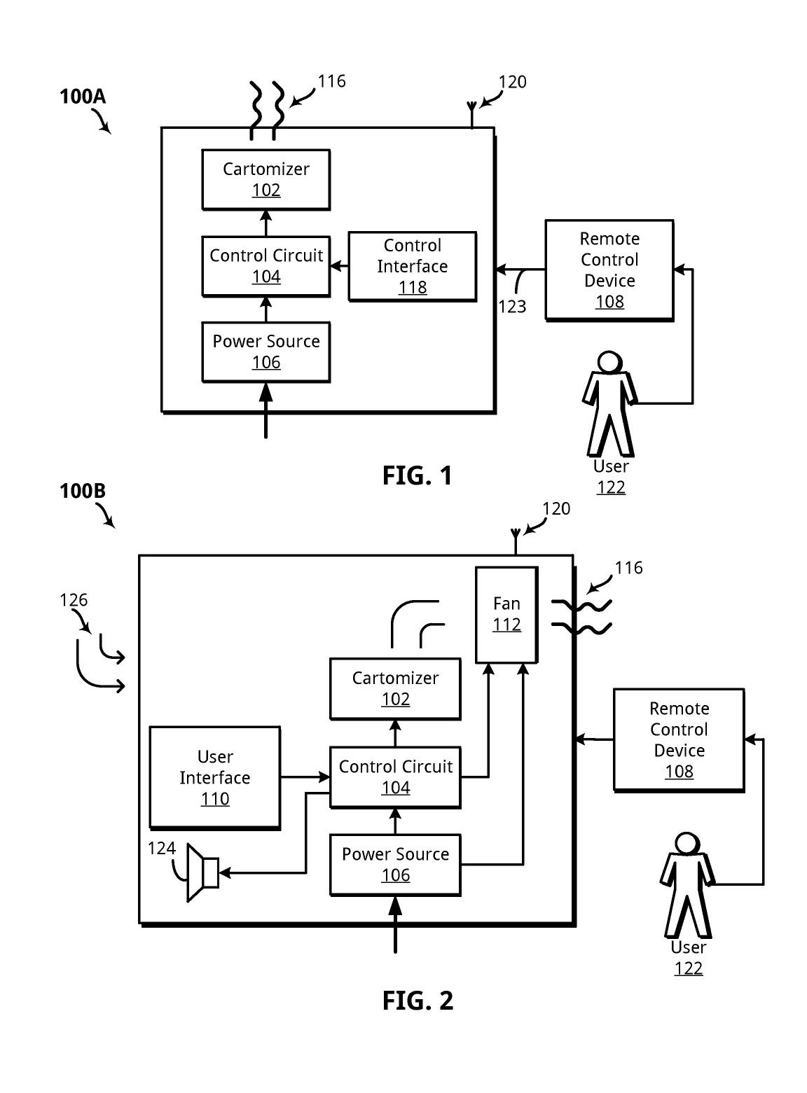

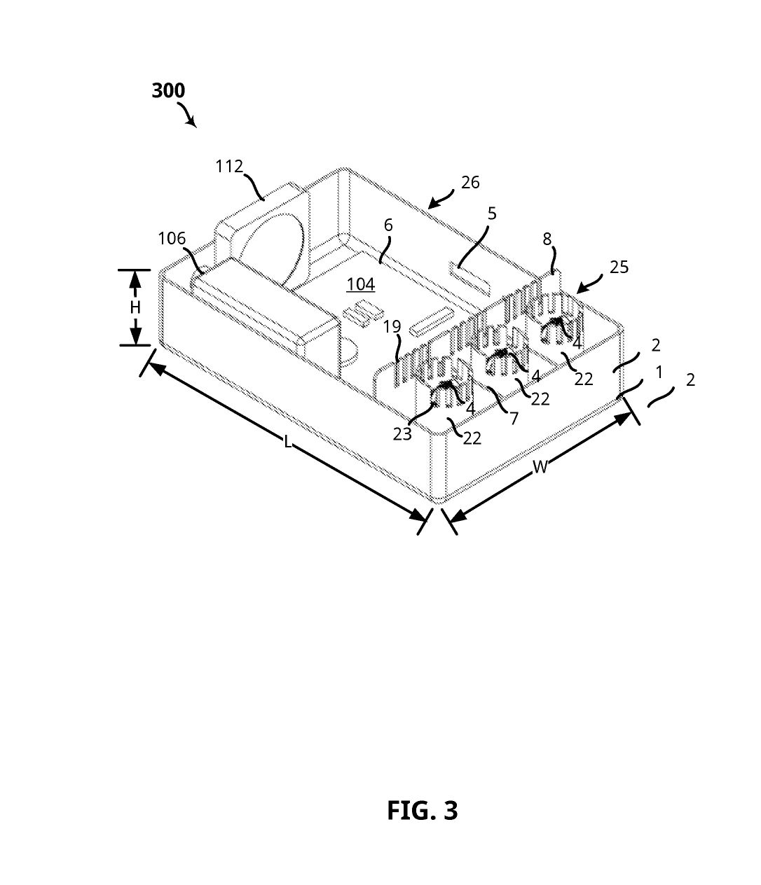

Referring now to the Figures, FIGS. 1 and 2 illustrate block diagrams of fragrance delivery devices consistent with embodiments of the present disclosure. FIGS. 3-9 illustrate a specific embodiment of a fragrance delivery device, and are discussed in greater detail below. Fragrance delivery devices consistent with embodiments of the present disclosure may have various sizes and configurations, and are not necessarily limited to the specific embodiments described and illustrated herein.

FIG. 1 illustrates one example fragrance delivery device 100A, and includes a cartomizer 102, a control circuit 104, and a power source 106 located within a housing. The fragrance delivery device 100A can further include a control interface 118 and a wireless radio receiver circuit 120, or radio. Although the control circuit 104, power source 106, control interface 118 and radio 120 are shown as separate components, this disclosure should not be construed as limited in this regard. For example, one or more components may be integrated into the same chip or chip(s). In addition, it should also be noted that communication between components within the fragrance delivery device 100A may be physical, such as through a bus or other electrical connection, or through wireless communication such as Bluetooth, ZigBee, Wi-Fi, or other suitable wireless approaches. Each of the components of the fragrance delivery device 100A will be discussed in turn.

Power source 106 may comprise a battery (not shown) or use an external power supply to supply power, or a combination of both. For example, the power source 106 may include a Universal Serial Bus (USB) port configured to receive a USB connector. Some such example port types include, for instance, USB A-Type, USB B-Type, Micro-USB A, Micro-USB B, Micro-USB AB, USB Mini-b (5-pin), USB Mini-b (4-pin), USB 3.0 A-Type, USB 3.0 B-Type, USB 3.0 Micro B, just to name a few. The USB connector may provide power to the fragrance delivery device 100A during use, or for recharging a battery, or both. In another example, the power source 106 may include an inductive charging system that includes a secondary coil that, when brought into proximity of a primary charging coil, receives power through a resulting electromagnetic field. Such inductive charging may also charge a battery of the power source 106. In still other examples, the power source 106 can include power conversion circuitry that may receive alternating current (AC) or direct current (DC) from a standard wall outlet, or from an external adapter that converts AC to DC. In still yet further examples, the power source 106 may comprise a photovoltaic cell to derive power from solar energy.

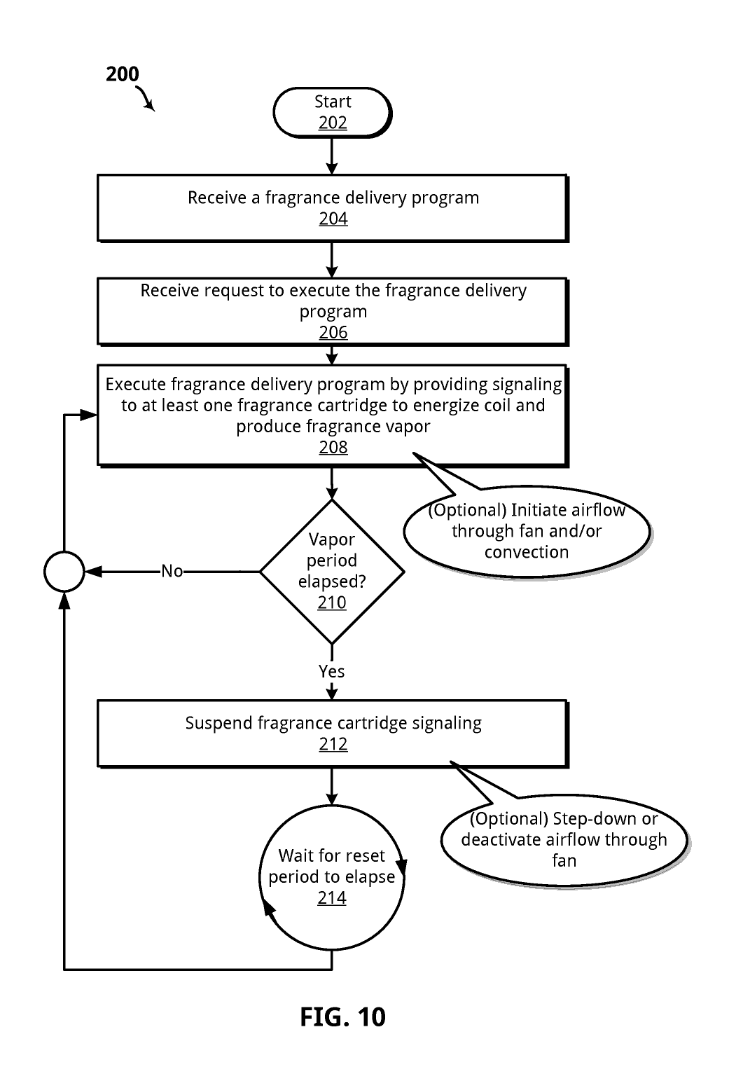

The control circuit 104 can comprise hardware, or a combination of hardware and software/firmware. The control circuit 104 may comprise, for example, an application-specific standard product (ASSP), a general-purpose processor (e.g., an x86 processor), an application-specific integrated circuit (ASIC), a field-programmable gate array (FPGA), a system-on-chip (SoC) device, a purpose-built silicon, discrete components, or any combination thereof. In an embodiment, the control circuit 104 may include a plurality of machine-readable instructions that, when executed, cause the method 200 of FIG. 10 to be carried out. Alternatively, or in addition to machine-readable instructions, circuitry of the control circuit 104 may perform all, or a portion, of the method 200. For instance, the method 200 may include using a timer to provide signaling to cartomizers (or cartridges, as discussed further below). Such timers can comprise, for example, a hardware clock (not shown) within the control circuit 104.

The control interface 118 may support so-called headless operation of the fragrance delivery device 100A through, for example, receiving user input via the radio 120 to adjust operation and programming of the fragrance delivery device 100A. To this end, the control interface 118 can include a radio 120 capable of receiving, for instance, Bluetooth connections. Other wireless protocols are also within the scope of this disclosure and may include, for example, ZigBee, inductive data transfer via a secondary coil within the control circuit 104, Wi-Fi, or other suitable wireless protocols. In the event a secondary coil is utilized, that same secondary coil may enable data transfer in addition to the power transfer through inductive coupling as discussed above. Note that the radio 120 may reside generally external to the housing, as shown, or may reside internally within the housing. The control interface 118 may also support adjustments to the operation/reprogramming of the fragrance delivery device through a wired connection, such as a universal serial bus (USB) connection. In any such cases, the data communication link 123 comprises those wired and/or wireless connections.

In any event, the control circuit 104 may receive input from the user 122 through the control interface 118, or through a remote control device 108, or both. The remote control device 108 can comprise, for example, a smartphone, laptop, personal heads-up display, or other such computing device. The remote control device 108 may communicate with the control interface 118 using for example, the data link 123 through radio 120. Alternatively, or in addition to a wireless data link, the remote control device 108 may communicate with the control interface 118 using a wired connection via data link 123, such as a USB data link. As discussed above, numerous data protocols are within the scope of this disclosure, and the control interface 118 is not necessarily limited to just one type. Moreover, the control interface 118 may support two or more such protocols, depending on a desired configuration.

In an embodiment, the control interface 118 includes hardware buttons, or other similar devices capable of receiving user input. In this embodiment, wireless operation through radio 120, discussed above, is optional. In any event, a liquid crystal display (LCD) may reside on or otherwise integrate with the housing, while a controller for the LCD may reside within the housing.

As previously discussed, the cartomizer 102 generally includes a reservoir for containing a fragrance liquid, a wick for receiving the fragrance liquid, and a heating coil around at least a portion of the wick for heating the fragrance liquid to produce the fragrance vapor. One specific example of a cartomizer cartridge is discussed with greater detail below.

In an embodiment, the fragrance delivery device 100A comprises a plurality of fragrance delivery programs. The fragrance programs may comprise a plurality of machine-readable instructions that when executed by at least one processor (e.g., within the control circuit 104) cause a fragrance delivery process to be carried out. These fragrance programs, in a general sense, govern fragrance delivery operations of the fragrance delivery device 100A. In more detail, the fragrance delivery programs govern an operational schedule that controls the frequency of fragrance delivery, and target characteristics for the fragrances delivered. By way of analogy, each fragrance delivery program may represent a musical score or symphony that allows the fragrance delivery device 100A to deliver specific fragrances in a controlled manner, much like musical notes, with a timing scheme that mitigates or otherwise eliminates olfactory fatigue. Accordingly, each fragrance delivery program includes one or more predetermined configuration parameters that provide fine-grain control of fragrance characteristics during fragrance delivery. Such characteristics may include, for example, the interval between producing a particular fragrance vapor, and the intensity of the fragrance vapor produced.

In addition, other such characteristics may include a target fragrance, with that target fragrance comprising a mixture of two or more fragrances. Thus, in accordance with a selected fragrance delivery program, the fragrance delivery device 100A may activate and combine fragrances to deliver those target fragrances with predetermined characteristics. The fragrance delivery device 100A may perform on-the-fly fragrance adjustment, in part, through natural convection caused by exposing the reservoir chamber of the cartomizer 102 to air, moving ambient air through the fragrance delivery device 100A, and by varying the length of a time a coil is energized/heated to produce fragrance vapor, or by any combination thereof.

In any event, the control interface 118 may receive input from user 122 via data link 123 and adjust a preprogrammed fragrance delivery program used to produce fragrance vapor 116 in a controlled/intermittent manner. For example, the fragrance delivery device 100A may get activated and controlled by the remote control device 108. Such devices can download an application, or so-called “app,” and use the same to make these adjustments or otherwise reprogram the behavior of the fragrance delivery device 100A. Additional operations of such an application may include, for example, executing a selected fragrance delivery program, adjusting one or more configuration parameters of a particular fragrance delivery program, downloading onto the fragrance delivery device 100A a new fragrance delivery program, ending performance of a selected fragrance delivery program to stop production of fragrance vapor 116, and establishing a playlist such that two or more fragrance delivery programs execute in sequential or random order, just to name a few. Alternatively, or in addition to remote configuration capabilities, the control interface 118 may provide the user input to the control circuit 104 to cause similar operations to occur locally.

In an embodiment, the adjusted programs/configuration parameters get stored in non-volatile memory of the control circuit 104 (not shown), such as Flash memory, and allows the fragrance delivery device 100A to retrieve previously stored data in the event of power loss or shutdown. In other cases, this data gets stored in volatile memory, such as static random access memory (SRAM).

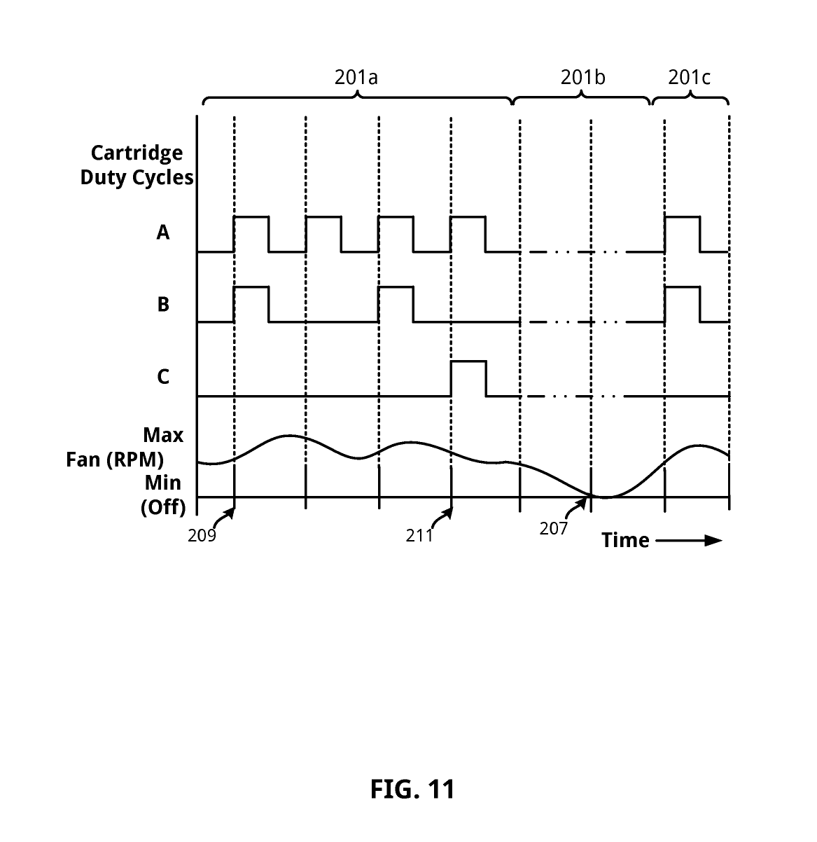

During operation, the fragrance delivery device 100A can execute a particular fragrance delivery program to cause the output of fragrance vapor 116. In an embodiment, the control circuit 104 commences fragrance delivery through a control scheme that selectively energizes one or more coils within the cartomizer 102, in accordance with a selected fragrance delivery program. For example, the control scheme may include using pulse width modulation (PWM) to convert a DC voltage from the power source 106 into a series of pulses, with the pulse duration, or period, being directly proportional to the value of DC voltage. The control circuit 104 can use, for example, a 555 integrated circuit (IC) to provide a clock mechanism for the PWM signal. In some cases, the duty cycle of the PWM signal can range from 0% to 100%. In addition, the selected duty cycle of the PWM signal can introduce gaps or intervals in fragrance presentation such that scent stimulus gets staggered, and thus allows the olfactory system to “reset”. So a user, or users, can continue to experience the fragrance vapor 116 without their brain's natural mechanisms preventing the enjoyment of that scent. One such specific example of using PWM signaling to produce fragrance vapor is discussed further below with regard to FIG. 11. However, other approaches to selectively energizing the coils will be apparent in light of the present disclosure, and the specific PWM signaling examples discussed herein should not be construed as limiting.

As discussed further below, and in an embodiment, the fragrance delivery device 100A can include 2 or more removable cartomizer cartridges, with each cartridge having its own wick, reservoir, and electrical contacts. The control circuit 104 can independently control each cartomizer cartridge to mix one or more aromas produced by the same to produce a target fragrance. In some cases, the control circuit 104 provides a different PWM signal to each cartridge via their respective electrical contacts. Thus, the control circuit 104 may independently control each cartomizer cartridge by, for example, providing varying duty cycles to toggle or otherwise stagger produced fragrance vapor 116 and fragrance intensity. In addition, the fragrance delivery device 100A can use variable convection to adjust or otherwise manipulate the experience a user(s) has during presentation of a fragrance. In some cases, a fan may provide this variable convection. One such example fragrance delivery device including a fan is discussed below with regard to FIG. 2.

Now referring to FIG. 2, another example fragrance delivery device 100B is shown, in accordance with an embodiment of the present disclosure. The example fragrance delivery device 100B is substantially similar to that of the example fragrance delivery device 100A of FIG. 1, but with the addition of a user interface 110, a fan 112, and an optional speaker 124. As should be appreciated, the aspects and embodiments discussed above with regard to the fragrance delivery device 100A are equally applicable to the fragrance delivery device 100B.

Also as discussed above, an LCD or other suitable user input device can comprise the control interface 118. In this embodiment, the user interface 110 is one such specific example implementation of a specialized control interface 118, and may visually represent a graphical user interface configured to receive user input (e.g., via a touch screen, hardware buttons, or other similar device).

Alternatively, or in addition to the user interface, a speaker 124 may aurally present information to the user 122. That information can include, for example, an audible beep or prompt that alerts the user 122 of, for instance, user input being received and acknowledged, configuration changes being applied, and shutdown and power-on events, just to name a few. As should be appreciated, the speaker 124 may advantageously support headless operation, as discussed above with regard to FIG. 1, because a user interface is not necessarily required when beeps and/or voice prompts (e.g., “Fragrance X selected”) allow the user 122 to effectively configure the fragrance delivery devices 100A and 100B.

The fan 112 may comprise a fan suitably sized to fit into the housing of the fragrance delivery device 100B. In some cases, the fragrance delivery device 100B is designed to be worn by user 122 or otherwise include a small footprint. In these cases, the fan 112 may comprise a micro fan having, for example, a length of 15 mm, a width of 15 mm, and a depth of 4 mm, which is approximately the size of a dime. It should be appreciated that other micro fan dimensions are also within the scope of this disclosure. In other cases, the fan 112 may comprise a standard, or non-micro fan, capable of producing airflow sufficient for circulating and/or expelling produced fragrances, and also for creating cross flow that allows ambient air 126 to enter the housing. Some examples of sufficient airflow may be relative to housing size, but in general may include at least 0.1 cubic feet per minute (cfm) or greater of air flow. Note that the fan 112 is not necessarily required to cause airflow in and out of the housing. For example, energizing the heating coil can cause natural convection that may provide sufficient airflow to produce fragrance vapor 116 at a perceivable level while people are within proximity of the fragrance delivery device 100A or 100B. Moreover, movement of the fragrance delivery device 100A or 100B, such as while worn by user 122, may cause ambient air 126 to enter the housing and, ultimately, push the fragrance vapor 116 from the housing.

Now referring to FIGS. 3-9, one specific example of a fragrance delivery device 300 is shown. As will be appreciated, the fragrance delivery device 300 may comprise the fragrance delivery devices 100A or 100B, depending on a desired configuration. However, it should be noted that the fragrance delivery device 100A and 100B are not necessarily mutually exclusive embodiments, and numerous combinations and permutations will be apparent in light of this disclosure. In any event, and as shown in FIG. 3, the fragrance delivery device 300 includes a base 1 having a length L and a width W, and sidewalls 2 having a height H, with the base 1 and sidewalls 2 forming a housing. As shown in FIG. 7, the housing may further include covers 3 and 11. The housing includes a cartomizer region 25, a control region 26, a printed circuit board (PCB) 6, power source 106, and fan 112. In addition, the housing can also include one or more data ports, such as USB port 5.

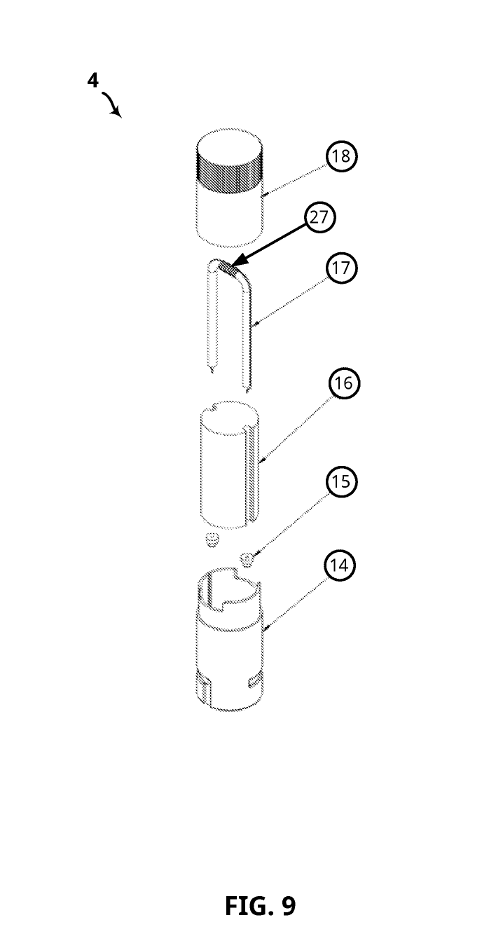

In more detail, the cartomizer region 25 includes one or more removable cartomizer cartridges 4 disposed within cams 22. In an embodiment, the cams 22 include a vented top portion 23. As shown, the cartomizer region 25 includes three removable cartomizer cartridges 4, positioned adjacent one another. The cartomizer cartridges 4 may lock into the base using, for example, a bayonet type locking mechanism, or another mechanism that attaches the cartridges 4 to the base 1. Each cam 22 may further include electrical contacts allowing the control circuit 104 to electrically couple to the cartomizer cartridges 4, and control production of fragrance vapor 116. In this embodiment, a wall section 7 separates adjacent cams 22, and forms cartomizer chambers for each cartomizer cartridge 4. The wall section 7 may extend from the base 1 to the top cover 11, or some distance short of the top cover 11. Another wall section 8 separates the cartomizer region 25 from the portion of the fragrance delivery device 300 that includes the power source 106, and the fan 112. The wall section 8 may include vents 19 that allow ambient air 126 to enter the cartomizer region 25. One specific example embodiment of a removable cartomizer cartridge is discussed in greater detail below with reference to FIG. 9. In an embodiment, the cartomizer region 25 includes one or more sensors located proximate to each of the cartomizer cartridges 4 to measure qualities of the fragrance vapor produced for monitoring purposes.

The fragrance delivery device 300 also includes a control region 26 including control circuit 104. The control circuit 104, in this embodiment, includes PCB 6 with one or more integrated components. Some such integrated components may include, for example, the control interface 118, the user interface 110, the speaker 124, the radio 120, circuitry to receive power from the power source 106, circuitry to control the removable cartomizer cartridges 4, and circuitry to drive and control the fan 112. To this end, some scenarios discussed herein reference the control circuit 104 in a manner that may encompass functionality provided through these integrated components. This embodiment of the fragrance delivery device 300 further includes an internal fan 112. As discussed above, natural convection may provide sufficient air movement, and so the internal fan 112 is not necessarily required. Note that a combination of the two may further enhance a user's perception and enjoyment of the fragrance vapor 116.

In some cases, the fragrance delivery device 300 may include multiple internal fans 112. Alternatively, or in addition to the internal fan 112, the fragrance delivery device 300 may include other types of air movement devices such as, for example, a bladeless fan assembly.

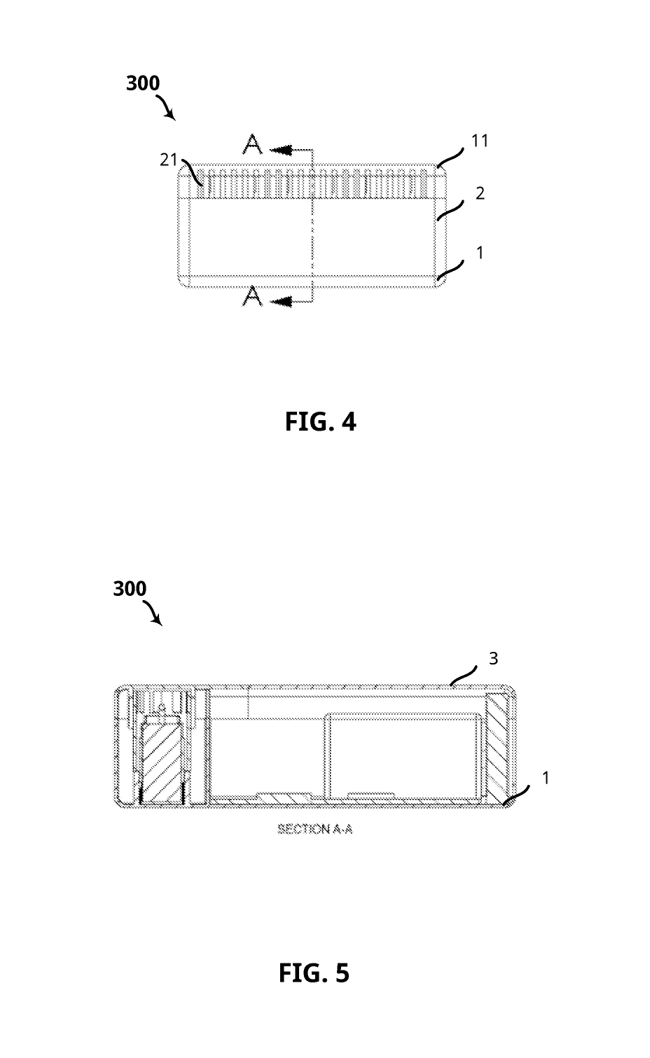

Referring to FIG. 4, shown are additional aspects of the example fragrance delivery device 300 of FIG. 3. In particular, FIG. 4 shows the base 1 and the top cover 11 covering the cartomizer region 25. In addition, the top cover 11 includes vents 21 configured to allow the fragrance vapor 116 to escape from the housing. The housing may include the vents 21 at different locations (e.g., on sidewall 2), and those locations are not necessarily limited to the top cover 11. FIG. 5 shows a side cross-sectional view of the fragrance delivery device 300 taken along line A-A in FIG. 4.

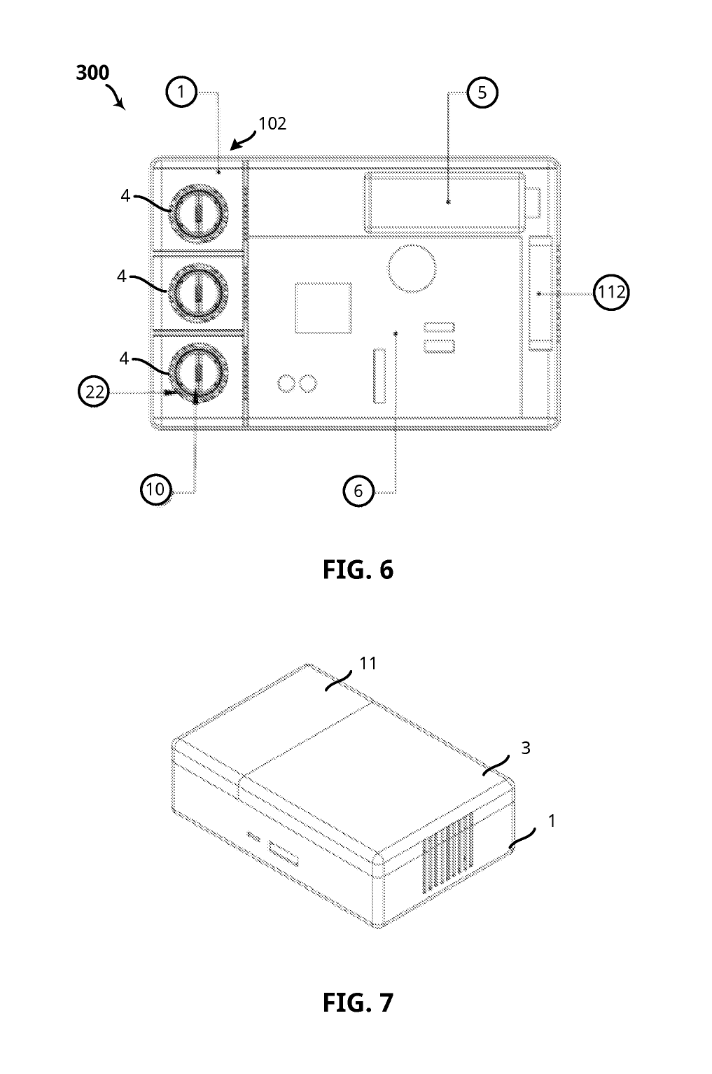

Now referring to FIG. 6, a top view of the fragrance delivery device 300 of FIG. 3 is shown, in accordance with an embodiment of the present disclosure. As shown, each of the removable cartomizer cartridges 4 includes a cartridge assembly portion 10. As discussed further below with regard to the embodiment of FIG. 9, the cartridge assembly portion 10 includes electrical contacts, a reservoir, a wick, and a cartridge body. This means that a user can expose the cartomizer cartridge region 25 and, for example, replace one or more cartomizer cartridges 4.

In an embodiment, a user can insert cartomizer cartridges 4 by removing top cover 11, inserting a cartomizer cartridge into a receptacle located in cams 22, and rotating the cartridge to lock it into place and make an electrical contact. In some cases, cartridges include a protective top cap (e.g., top cap 18 of FIG. 9), that gets removed prior to putting the top cover 11 back into position. In any event, the user can place the top cover 11 back onto the cartomizer region 25. In some cases, the top cover 11 locks the cartomizer cartridges 4 in place, and protects wicks from damage.

FIG. 7 shows a perspective view of the fragrance delivery system 300 of FIG. 3 with installed top covers 3 and 11, in accordance with an embodiment of the present disclosure. In this embodiment, the top cover 11 and 3 are each separately removable to expose the cartomizer region 102 or to expose the region having the power source 106 and the PCB 6.

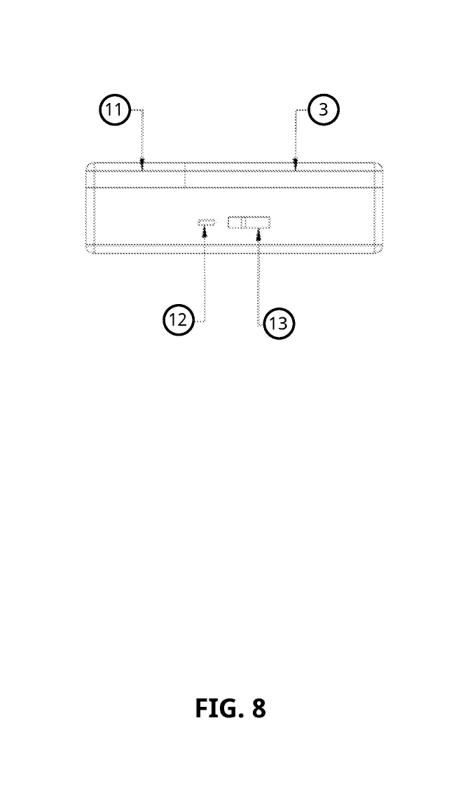

FIG. 8 shows side view of the covered fragrance delivery device of FIG. 7, in accordance with an embodiment of the present disclosure. As shown, the covered fragrance delivery device includes a data/power port 12 comprising a micro USB port, and a second data/power port 13 comprising a standard size USB port. The fragrance delivery device of FIG. 7 can include a different number of ports, and the number shown in FIG. 8 should not be construed as limiting. The control circuit 104 can use such ports to derive power, and in some cases, receive user input to program/adjust operation of fragrance delivery device.

Also note that fragrance delivery devices consistent with the present disclosure are not limited to three cartomizer cartridges. In some cases, a fragrance delivery device includes a single cartomizer cartridge. In other cases, a fragrance delivery device includes four or more cartridges. Such devices may be worn by a user, or configured to be table-top devices.

FIG. 9 shows an exploded view of one example cartomizer cartridge 4 for use in a fragrance delivery device, in accordance with an embodiment of the present disclosure. The cartomizer cartridge 4 may include a cartridge body 14, a wick 17 with a heating coil 27 wrapped around at least a portion thereof, a reservoir 16, and electrical contacts 15 configured to electrically couple to ends of the heating coil.

In more detail, the cartridge body 14 may be configured to be removably engaged within the housing (e.g., using cams 22), with a bayonet type locking mechanism, such as shown. In other embodiments, the cartridge body 14 can include a thread or other suitable locking mechanism. The cartomizer cartridge 4 may further include a cap 18 sized and configured to removably engage with the cartridge body 14. The heating coil 27 may comprise a resistive heating coil capable of generating heat when energized by an electrical current. In some cases, the heating coil 27 comprises a resistive nichrome wire coil. Other suitable heating coils are also within the scope of this disclosure, and will be apparent in light of this disclosure.

In an embodiment, the cartomizer cartridge 4 includes a machine-readable identifier that may allow the fragrance delivery device to determine the presence of a particular cartridge, and may allow the fragrance delivery device to identify the particular fragrance that cartridge can produce. In some cases, the cartomizer cartridge 4 provides this via a miniaturized circuit that can provide a machine-readable signal, or by other electrical/mechanical mechanisms, as will be appreciated in light of this disclosure.

The reservoir 16 may hold a desired material, also generally referred to as fragrance liquid, to be vaporized into fragrance vapor 116. Each fragrance cartridge may include a different type of fragrance liquid, with each fragrance liquid providing a different aroma. The wick 17 may absorb the fragrance liquid from the reservoir 16 through capillary action. The fragrance delivery device may then energize coil 27, thus heating coil 27, which then may cause the liquid fragrance to vaporize into fragrance vapor 116. The fragrance vapor 116 then enters the cartomizer chamber (e.g., cartomizer region 25) and ultimately escapes the chamber by way of natural convection, air displacement by a fan, or both.

As discussed above, an embodiment of the present disclosure includes a fragrance delivery device configure to execute one or more fragrance delivery programs, similar to a playlist configured to play multiple songs/tracks on a portable music player. One such example method 200 of executing a fragrance delivery program is shown in FIG. 10. Method 200 may be executed by, for example, the control circuit 104 of fragrance delivery devices 100A and 100B. The method 200 begins in act 202.

In act 204, the fragrance delivery device receives a fragrance delivery program. This can include, for example, the fragrance delivery device retrieving the fragrance delivery program from a memory, such as from a Flash memory device or other non-volatile memory. The fragrance delivery device may retrieve the program based on, for example, a local or remote user-generated request to retrieve the program. In response, the fragrance delivery device may load the fragrance delivery program into for example, random access memory (RAM) to execute the program. As previously discussed with regard to FIG. 1A, computing devices using an “app” can download or otherwise transfer preprogrammed fragrance delivery programs onto the fragrance delivery device via communication link 123.

In act 206, the fragrance delivery device receives a request to execute the fragrance delivery program to cause the production and output of fragrance vapor 116. In some cases, a remote device may initiate the request via the communication link 123, or through local user input via the graphical user interface 110, for example. In other cases, the request is automated based on a pre-programmed schedule, with that pre-programmed schedule being user-defined, or provided during manufacturing, and establishing what fragrance programs to execute and how often to execute them (e.g., hourly, daily, weekly, and so on).

In act 208, the fragrance delivery device executes the fragrance delivery program and provides a signal to the at least one cartomizer cartridge 4 in order to controllably energize a coil 27 within each cartomizer cartridge 4, and to cause output of fragrance vapor 116. In an embodiment, the signal comprises a PWM signal. As discussed below with regard to FIG. 11, the PWM signal may be controllably provided to each respective cartomizer cartridge 4 operatively coupled to the control circuit 104 within the fragrance delivery device.

FIG. 11 illustrates one example timing diagram for a plurality of PWM signals controllably provided to a plurality of cartomizer cartridges 4 in order to cause vapor production. As shown in this embodiment, the example timing diagram represents a plurality of PWM signals, A, B and C, over a plurality of time periods 201a, 201b and 201c. Each dotted horizontal line represents, for example, a signal output by a hardware or software clock (e.g., the rising edge of a square wave provided by one clock cycle of a 555 timer) provided by the control circuit 104. Note that while three PWM signals are depicted, some embodiments herein contemplate a fragrance delivery device having as few as one cartomizer cartridge, or more than three cartridges, depending on a desired configuration. To this end, the number of PWM signals shown should not be construed as limiting the present disclosure.

The control circuit 104 can provide the hardware/software and logic (e.g., gates) that may vary the duty cycle of the PWM signals provided to each of the cartomizer cartridges 4, thus changing when each cartomizer cartridge produces vapor, and the intensity of that vapor. The fragrance delivery device can provide each of PWM signals A, B and C to different cartomizer cartridges, such as the three shown in FIGS. 3 and 6. For example, the A PWM signal includes a first duty cycle of approximately 100% during period 201a, which causes the particular cartomizer cartridge receiving that signal to produce its vapor at a maximum output rate/intensity. In contrast, the B PWM signal during the same period 201a includes a second duty cycle of approximately 50%, thus resulting in a proportional vapor output rate/intensity of 50%. Likewise, the “C” PWM signal during period 201a includes a third duty cycle of approximately 25%, thus resulting in a proportional vapor output rate/intensity of 25%.

Note that control circuit 104 can provide signals with different duty cycles to each cartomizer cartridge, as shown, or provide signals with the same duty cycles to two or more cartridges. As previously discussed, the fragrance delivery device orchestrates fragrance production similar to musical notes. Thus, the PWM signals provided to each of the cartomizer cartridges may vary to provide fragrance vapor having the characteristics that comport with the executed fragrance programs. To this end, the fragrance delivery device can mix the fragrance vapor of two or more cartomizer cartridges to achieve a desired aroma. For instance, at clock cycle 209, the A and B signal may cause their corresponding cartomizer cartridges to produce, essentially, an aroma that is A+B. Likewise, at clock cycle 211, the A and C signal may cause their corresponding cartomizer cartridges to produce, essentially, an aroma that is A+C. In other examples, A, B and C signals may cause their corresponding cartomizer cartridges to produce essentially, an aroma that is A+B+C. Note that if the fragrance delivery device includes additional fragrance cartridges, then signals to those cartridges (e.g., a D and E signal) may be utilized. Further note that a single signal may be provided to pairs or other groupings of cartridges. For instance, the A signal may be provided to two or more fragrance cartridges, while the B signal may be provided to two or more different fragrance cartridges. Other combinations and variations will be apparent in light of this disclosure.

It should be appreciated that duty cycles may change during execution of a fragrance delivery program. For example, the A PWM signal may shift from 100% to 25%, and then back again. In another example, both the A and B PWM signals may shift to 0%, while the C PWM signal shifts to 100% during a same period. These shifts may take effect for N number of clock cycles, depending on the characteristics defined within the particular fragrance program being executed. In any event, it should be appreciated in light of this disclosure that numerous variations and permutations are possible with regard to the signaling used, and the provided examples should not be construed as limiting.

Continuing with FIG. 11, the example timing diagram also shows a signal provided to a fan, such as fan 112, relative in time to the PWM signals. The fan signal includes a minimum and maximum value, with the maximum value representing the maximum RPMs possible for the particular fan 112. As shown, during period 201a, the fragrance delivery device may adjust RPMs to control air displacement and circulation. This control allows the fragrance delivery device to selectively adjust airflow to enhance a user's sensory experience. In an embodiment, a sensor located proximate to the removable cartomizer cartridges 4 provides a feedback loop that allows the fragrance delivery device to make such airflow adjustments.

In an embodiment, the fragrance delivery device may increase air displacement via fan 112 during the period 201a. Nearing the end of period 201a, the fragrance delivery device may step-down the fan's RPMs in favor of, for example, natural convection created by exposing ambient air to the cartomizer cartridges. In any event, as the fragrance delivery device approaches the period 201b, generally referred to herein as the “reset” period, the fragrance delivery device can reduce the airflow, and thus, begin the process of lowering the amount of fragrance vapor a user is experiencing in anticipation of the reset period 201b.

As shown in period 201b, the duty cycle of each of the PWM signals A, B, and C is at or near 0%. The reset period 201b may suspend vapor production and allow a user(s) to avoid potential olfactory fatigue. A predetermined amount of time, generally referred to herein as a reset period, elapses prior to the fragrance delivery device producing additional fragrance vapor. Once the reset period elapses, and during period 201c, the fragrance delivery device provides PWM signaling to again produce fragrance vapor. Note that at 207, the fan 112 may continue to displace air during at least a portion of the reset period 201b. In such cases, the fan 112 may continue to push residual fragrance vapor from the cartomizer cartridges even after their coils are no longer energized. Such a feature may, for example, conserve power consumption by the fragrance delivery device.

Returning to FIG. 10, and in act 210, the fragrance delivery device determines if the period of time fragrance vapor has been output exceeds a predefined period. Recall that olfactory fatigue can occur shortly after exposure to a particular scent. To this end, the predefined period may comprise a range of 1 second to 3 minutes, for example. In any event, if the predefined period has elapsed the method 200 continues to 212. Otherwise, the method 200 returns to act 208 to continue providing signaling in accordance with the fragrance delivery program executed in act 206.

In act 212, the fragrance delivery device suspends providing a signal to the cartomizer cartridges 4. In some cases, this also includes stepping down or turning off air flow provided by the fan 112. In act 214, the fragrance delivery device waits for a predefined reset period to elapse. In some cases, the predefined reset period may comprise a range of, for example, 10 seconds to 4 minutes. The method 200 returns to act 208 once the reset period has elapsed. Acts 208-214 continue until otherwise interrupted by, for example, shutdown of the fragrance delivery device, user input selecting a different fragrance delivery program for execution, user input requesting the fragrance delivery device stop the presently executed fragrance delivery program, or the presently executed fragrance delivery program has a scheduled end (similar to a music track ending). In some cases, the user can pause and continue execution of the fragrance delivery program executed in act 206, similar to pausing and resuming songs/tracks on a music player.

Accordingly, a fragrance delivery device, consistent with embodiments of the present disclosure, allows different fragrances to be delivered selectively, with adjustable intensity, and intermittently to mitigate the effects of olfactory fatigue.

In one aspect, a fragrance delivery device is disclosed. The fragrance delivery device comprising a housing, at least one cartomizer located in the housing, the cartomizer including a reservoir for containing a fragrance liquid, a wick for receiving the fragrance liquid, and a heating coil around at least a portion of the wick for heating the fragrance liquid and producing fragrance vapor, and control circuitry located in the housing, the control circuitry configured to receive a user input and configured to generate a control signal, in response to the user input, for controllably energizing the heating coil to intermittently heat the fragrance liquid and intermittently produce the fragrance vapor.

The control circuitry may be configured to generate the control signal using pulse width modulation to adjust intensity of producing the fragrance vapor. The at least one cartomizer may include at least one removable cartomizer cartridge. The control circuitry may be configured to generate control signals for each of the cartomizer cartridges to selectively control producing the fragrance vapor from one or more of the cartomizer cartridges. The control circuitry may configured to store different fragrance delivery programs in a memory.

In an embodiment, the fragrance delivery device may further comprise a power source, the power source being at least one of a battery, an external power supply, and a photovoltaic cell. The power source may comprise a universal serial bus connection to an external power source. In another embodiment, the control circuitry may be configured to communicate wirelessly with a remote control device, the remote control device being a mobile computing device.

In an embodiment, the fragrance delivery device may further comprise a user interface coupled to the control circuitry for providing the user input. In an embodiment, the fragrance delivery device may further comprise a fan for circulating the fragrance vapor produced by the at least one cartomizer. The control circuitry may be configured to control a speed of the fan. In another embodiment, the fragrance delivery device includes an optical sensor configured to detect a rate of fragrance vapor creation in proximity to the heating coil, and wherein the control circuitry is configured to receive an output from the optical sensor. The housing may be configured to be worn by a user.

In another aspect, a cartomizer cartridge for use in a fragrance delivery device is disclosed. The cartomizer cartridge comprising a cartridge body including an engagement mechanism for being removably engaged within a housing in a fragrance delivery device, a reservoir within the cartridge body for storing a fragrance liquid, a wick extending around at least a portion of the reservoir for receiving the fragrance liquid, a heating coil around at least a portion of the wick, wherein ends of the heating coil extend to one end of the cartridge body, and electrical contacts electrically connected to the ends of the heating coil and configured to engage corresponding electrical contacts in the fragrance delivery device. The cartomizer cartridge may further comprise a removable cap for removably engaging the cartridge body.

In yet another aspect, a method for providing a signal to a cartomizer cartridge for use in a fragrance delivery device is disclosed. The method comprising generating, by a control circuit, a first signal, the first signal having a first duty cycle, generating, by the control circuit, a second signal, the second signal having a second duty cycle, and providing the first and second signal to a first and second cartomizer cartridge, respectively.

In an embodiment, the method further comprises receiving, by the control circuit, a fragrance delivery program, wherein the fragrance delivery program includes at least one characteristic; and adjusting at least one of the first and second duty cycles based on the at least one characteristic. In yet another embodiment, the method further comprises receiving, by a wireless radio circuit, user input, and adjusting at least one of the first duty cycle and second duty cycle based on the user input.

In an embodiment, the method further comprises activating, by the control circuit, a fan, the fan being configured to circulate air over at least one of the first and second cartomizer cartridges, and adjusting a fan speed of the fan based on a signal from a sensor located proximately to the first and second cartomizer cartridges. The first duty cycle of the first generated signal may be different than the second duty cycle of the second generated signal.

While the aspects and embodiments of the disclosure have been described herein, it is to be understood by those skilled in the art that this description is made only by way of example and not as a limitation as to the scope of the disclosure. Other embodiments are contemplated within the scope of the present disclosure in addition to the exemplary embodiments shown and described herein. Modifications and substitutions by one of ordinary skill in the art are considered to be within the scope of the present disclosure, which is not to be limited except by the following claims.

What is claimed is:

1. A fragrance delivery device comprising:
a housing;
at least one cartomizer located in the housing, the cartomizer including a reservoir for containing a fragrance liquid, a wick for receiving the fragrance liquid, and a heating coil around at least a portion of the wick for heating the fragrance liquid and producing fragrance vapor; and
control circuitry located in the housing, the control circuitry configured to receive a user input and configured to generate a control signal, in response to the user input, for controllably energizing the heating coil to intermittently heat the fragrance liquid and intermittently produce the fragrance vapor.

2. The fragrance delivery device of claim 1, wherein the control circuitry is configured to generate the control signal using pulse width modulation to adjust intensity of producing the fragrance vapor.

3. The fragrance delivery device of claim 1, wherein the at least one cartomizer includes at least one removable cartomizer cartridge.

4. The fragrance delivery device of claim 1, wherein the control circuitry is configured to generate control signals for each of the cartomizer cartridges to selectively control producing the fragrance vapor from one or more of the cartomizer cartridges.

5. The fragrance delivery device of claim 1, wherein the control circuitry is configured to store different fragrance delivery programs in a memory.

6. The fragrance delivery device of claim 1, further including a power source, the power source being at least one of a battery, an external power supply, and a photovoltaic cell.

7. The fragrance delivery device of claim 6, wherein the power source comprises a universal serial bus connection to an external power source.

8. The fragrance delivery device of claim 1, wherein the control circuitry is configured to communicate wirelessly with a remote control device, the remote control device being a mobile computing device.

9. The fragrance delivery device of claim 1, further comprising a user interface coupled to the control circuitry for providing the user input.

10. The fragrance delivery device of claim 1, further comprising a fan for circulating the fragrance vapor produced by the at least one cartomizer.

11. The fragrance delivery device of claim 10, wherein the control circuitry is configured to control a speed of the fan.

12. The fragrance delivery device of claim 1, further including an optical sensor configured to detect a rate of fragrance vapor creation in proximity to the heating coil, and wherein the control circuitry is configured to receive an output from the optical sensor.

13. The fragrance delivery device of claim 1, wherein the housing is configured to be worn by a user.

14. A cartomizer cartridge for use in a fragrance delivery device, the cartomizer cartridge comprising:
a cartridge body including an engagement mechanism for being removably engaged within a housing in a fragrance delivery device;
a reservoir within the cartridge body for storing a fragrance liquid;
a wick extending around at least a portion of the reservoir for receiving the fragrance liquid;
a heating coil around at least a portion of the wick, wherein ends of the heating coil extend to one end of the cartridge body; and
electrical contacts electrically connected to the ends of the heating coil and configured to engage corresponding electrical contacts in the fragrance delivery device.

15. The cartomizer cartridge of claim 14, further comprising a removable cap for removably engaging the cartridge body.

16. A method for providing a signal to a cartomizer cartridge for use in a fragrance delivery device, the method comprising:
generating, by a control circuit, a first signal, the first signal having a first duty cycle;
generating, by the control circuit, a second signal, the second signal having a second duty cycle; and
providing the first and second signal to a first and second cartomizer cartridge, respectively.

17. The method of claim 16, further comprising:
receiving, by the control circuit, a fragrance delivery program, wherein the fragrance delivery program includes at least one characteristic; and
adjusting at least one of the first and second duty cycles based on the at least one characteristic.

18. The method of claim 16, further comprising:
receiving, by a wireless radio circuit, user input; and
adjusting at least one of the first duty cycle and second duty cycle based on the user input.

19. The method of claim 16, further comprising:
activating, by the control circuit, a fan, the fan being configured to circulate air over at least one of the first and second cartomizer cartridges; and
adjusting a fan speed of the fan based on a signal from a sensor located proximately to the first and second cartomizer cartridges.

20. The method of claim 16, wherein the first duty cycle of the first generated signal is different than the second duty cycle of the second generated signal.
